(19) United States
(12) Patent Application Publication
(10) Pub. No.: US 2018/0235206 A1
(43) Pub. Date: Aug. 23, 2018

(54) COMPOSITIONS AND METHODS FOR STABILIZING DNA IN SALIVA SAMPLES

(71) Applicant: OASIS DIAGNOSTICS CORPORATION, VANCOUVER, WA (US)

(72) Inventors: MARY LAUGHLIN, PORTLAND, OR (US); ROBERT L BUCK, PORTLAND, OR (US); GERALD A THOMAS, PORTLAND, OR (US)

(73) Assignee: OASIS DIAGNOSTICS CORPORATION, VANCOUVER, WA (US)

(21) Appl. No.: 15/899,087

(22) Filed: Feb. 19, 2018

Related U.S. Application Data
(60) Provisional application No. 62/460,641, filed on Feb. 17, 2017.

(51) Int. Cl. A01N 1/02; C12Q 1/6806

(52) U.S. Cl. CPC A01N 1/0215; C12Q 2527/125; C12Q 1/6806

(57) ABSTRACT

A composition and method for stabilizing DNA in saliva at room temperature. The composition comprises a denaturing agent, a chelating agent, a biocide and trehalose in a gel formulation. Such a composition is re-hydrated upon contact with saliva. Also included are means for storing the composition.

### BACKGROUND

Biological samples are used for a variety of purposes from diagnostics to genetic analysis. However, most biological fluids such as blood, urine, and saliva contain a variety of substances that may degrade or sequester nucleic acids or otherwise hamper later analysis. Unless the samples are analyzed on the spot, the highly unstable nature of biological samples requires preservation immediately or shortly after collection in order to maintain the integrity and stability of the targeted materials of interest in the biological materials. Even with preservation, there is research showing that in common storage situations, low concentration samples still degrade and become otherwise unavailable (Smith S, Morin P A. Optimal storage conditions for highly dilute DNA samples: a role for trehalose as a preserving agent. J Forensic Sci. 2005 September; 50(5):1101-8.)

Generally, biological samples are preserved through refrigeration or dehydration. Typical refrigeration storage conditions require temperatures below 0° C., typically at about −20° C. or at −70° C. to −80° C. requiring special equipment which creates difficulties with collection in the field and transportation of the samples. During dehydration the samples are not immediately preserved, leaving nucleic acids in the samples susceptible to damage or chemical modification by enzymes in the sample. (Handbook of Nuclear Acid Purification, edited by Dongyou Lu, CRC Press 2009). Additionally, dehydration may decrease activity upon reconstitution and cause irreversible aggregation. Liquid buffers are of limited use for home-collection, as the untrained user may mishandle the device and spill or otherwise compromise the buffer. Dry pellets are useful, but also subject to mishandling. The same limitations apply to trained persons operating in field conditions, or in other difficult collection conditions.

The limited stability of the components of biological samples such as saliva requires special equipment, training and methods, limiting its collection to clinical settings and precluding remote and field collection. Accordingly, there is a need in the art for compositions and methods for storing biological samples for extended time periods while maintaining the biological activity.

### BRIEF SUMMARY

A method and composition for preserving deoxyribonucleic acid (DNA) in saliva at room temperature by contacting saliva with a semi-solid stabilizing composition comprising a chelating agent, a denaturing agent, a biocide and a sugar such as trehalose.

While any chelating agent may be used, in some embodiments, the chelating agent may be selected from ethylene glycol-bis(β-aminoethyl ether)-N,N,N′,N′-tetraacetic acid (EGTA), ethylenediamine tetraacetic acid (EDTA), cyclohexane diaminetetraacetate (CDTA), diethylenetriamine pentaacetic acid (DTPA), tetraazacyclododecanetetraacetic acid (DOTA), tetraazacyclotetradecanetetraacetic acid (TETA), or desferrioximine.

The denaturing agent may be selected from Tween, Triton, Nonidet, Igepal, Tergitol, sodium dodecyl sulfate (SDS), N-lauroyl sarcosine; diethyl glycol monoethyl ether (DGME), Myristyltrimethylammonium bromide, 3-[(3-cholamidopropyl) dimethylammonio]-1-propanesulfonate (CHAPS), Tri-methyl-tetradecyammonium chloride (TTAC), guanidine hydrochloride, guanidinium thiocyanate, sodium thiocyanate, sodium iodide, sodium perchlorate, TCEP (tris(2-carboxyethyl)phosphine or urea.

The biocide may be selected from 5-chloro-2-methyl-4-isothiazolin-3-one, sodium azide, 2-methyl-4-isothiazolin-3-one, thimerosal, or hypochlorite.

Further provided is a collection device for storing both the saliva and the semi-solid stabilizing composition where the collection device has a first region with a first open end for collection of the saliva and a second region with a second open end where the stabilizing composition is stored in a sealing element located in the second region of the collection device. After the saliva is collected, the collection tube is closed by inserting the first end of the sealing element in the first open end of the collection tube and agitating the scaled collection tube to combine the stabilizing formulation with the saliva forming a liquid mixture which preserves DNA in saliva at room temperature.

These and other embodiments, features and potential advantages will become apparent with reference to the following description.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an aspect of a collection device in accordance with one embodiment.

FIG. 2 illustrates an aspect of a collection device in accordance with one embodiment.

FIG. 3 illustrates an aspect of a collection device in accordance with one embodiment.

FIG. 4 illustrates an aspect of a collection device in accordance with one embodiment.

FIG. 5 is a chart depicting the inhibition of deoxyribonucleases (DNases) by the semi-solid formulation described herein in comparison to controls.

FIG. 6 is a chart comparing DNase activity in saliva samples at different temperatures on Day 5.

FIG. 7 is a chart comparing DNase activity on Day 12.

FIG. 8 is a chart depicting DNase activity on Day 19.

FIG. 9 is a chart depicting DNase activity on Day 30.

FIG. 10 is a gel comparing DNA degradation over time in preserved and control saliva.

FIG. 11 is a gel comparing DNA degradation over time in preserved and control saliva.

FIG. 12 is a gel comparing DNA degradation over time in preserved and control saliva.

FIG. 13 is a gel comparing DNA degradation over time in controls.

FIG. 14 is a gel comparing DNA degradation over time in preserved saliva.

FIG. 15 is a photograph showing bacterial growth after three days in saliva alone and treated in accordance with one embodiment.

FIG. 16 is a photograph showing yeast and mold growth after five days in saliva alone and treated in accordance with one embodiment.

FIG. 17 is a photograph showing bacterial growth after fifty-six days in saliva alone, saliva treated with control and saliva treated in accordance with one embodiment.

FIG. 18 is a photograph showing yeast and mold growth after fifty-six days in saliva alone, saliva treated with control and saliva treated in accordance with one embodiment.

### DETAILED DESCRIPTION

“About” in this context refers to +/−10% of the stated value or a chemical equivalent thereof.

“Biocide” in this context refers to a chemical substance or microorganism intended to destroy, deter, render harmless, or exert a controlling effect on any harmful organism by chemical means.

“Stabilizing formulation” in this context refers to semi-solid gel formulation containing a chelator, denaturing agent, biocide and sugar as described herein.

“Stabilized nucleic acid” in this context refers to the prevention of degradation of at least 50% of the initial high molecular weight DNA after storage at room temperature as determined by quantitation of the recovered gel band intensity by image analysis.

### DESCRIPTION

Provided herein are compositions and methods for stabilizing DNA in saliva samples at room temperature. Specifically described herein is a semi-solid stabilizing formulation which, when mixed with saliva, preserves the nucleic acids at room temperature under ambient conditions for extended periods of time. Such stabilization of DNA in a saliva sample is attained through the use of chelators, biocides, and denaturing agents. The chelator, biocide and denaturing agent are dried with trehalose or another sugar to form a semi-solid gel which may be rehydrated by mixing with a saliva sample, whereupon the DNA in the saliva is stabilized, allowing for storage and transportation of the sample without the need for freezing, lyophilizing or other cumbersome preservation methods.

Saliva is the product of three pairs of major salivary glands, parotid (PAR), submandibular (SM) and sublingual (SL), in addition to multiple minor salivary glands lying beneath the oral mucosa. It is an attractive diagnostic fluid as collection is simple, low-cost and non-invasive. While any method of saliva collection may be used, generally unstimulated collection is used to obtain an adequate sample. For example, in some embodiments the drool method of saliva collection is used. In the drool method, 10 minutes prior to collection of unstimulated saliva samples, individuals are asked to rinse orally with water and then asked to relax for 5 to 15 minutes. They are then seated in a bent forward position in an ordinary chair and asked to put their tongues on the lingual surfaces of the upper incisors and allow the saliva to drip into a container until the desired amount is collected. If a subject is unable to generate a sample of saliva, substances such as citric acid or sugar may be contacted with the buccal cavity to generate a reflex stimulation of saliva. In another embodiment, an individual may be asked to rinse their mouth and discard the liquid, then wait five minutes before expectorating into a collection device until the desired amount is collected. In further embodiments, the saliva collecting device may comprise an absorbent pad material that is made up of hydrophilic materials. Those skilled in the art know that absorbent pad materials may also include hydrophilic or hydrophobic components bound, or integrated into the material, such components being capable of modifying the absorption and release characteristics of the absorbent pad as well as the speed of uptake of the sample fluid under consideration.

In some embodiments, various saliva collection devices which may be used for stimulated or unstimulated saliva collection such as those described in U.S. Pat. No. 7,618,591 and U.S. Pat. No. 8,025,851, and U.S. patent application Ser. No. 15/360,701.

In some embodiments an adequate sample is about 1.5 mL to about 3 mL, preferably about 2.0 mL to about 2.5 mL. After collection, the saliva is mixed with the semi-solid stabilizing formulation comprising a chelator, denaturing agent, biocide and a sugar to create a DNA stabilized saliva sample which may be stored at room temperature.

DNA has a strong affinity for metal ions, some of which can catalyze the formation of reactive oxygen species as well as unwind DNA (Interaction of metal ions with polynucleotides and related compounds. XII. The relative effect of various metal ions on DNA helicity Gunther L. Eichhorn and Yong Ae Shin Journal of the American Chemical Society 1968 90 (26), 7323-7328 DOI: 10.1021/ja01028a024). The stabilizing formulation described herein comprises one or more chelators that can form complexes with metal ions to prevent them from binding to DNA, remove metal ions that have already bound to DNA, or bind to metal ions (e.g., Fe(II)/Fe(II) or Cu(I)/Cu(II)) strongly enough to inhibit their Lewis acid or redox cycling. Exemplary chelating agents for use within the formulations describe herein include, but are not limited to, ethylene glycol-bis(β-aminoethyl ether)-N,N,N′,N′-tetraacetic acid (EGTA), ethylenediamine tetraacetic acid (EDTA), cyclohexane diaminetetraacetate (CDTA), diethylenetriamine pentaacetic acid (DTPA), tetraazacyclododecanctetraacetic acid (DOTA), tetraazacyclotetradecanetctraacetic acid (TETA), and desferrioximine, or chelator analogs thereof. The amount of chelating agent may be from about 200 to about 500 mM of the formulation.

Deoxyribonuclcascs and ribonucleases are enzymes that break down DNA or RNA, respectively. The action of deoxyribonucleases and ribonucleases can be inhibited by denaturing agents that destroy the structures of these enzymes. The stabilizing formulation described herein comprises one or more denaturing agents including, but not limited to, anionic, cationic, zwitterionic or non-ionic detergents such as Tween, Triton, Nonidet, Igepal, Tergitol, sodium dodecyl sulfate (SDS) or N-lauroyl sarcosine; diethyl glycol monoethyl ether (DGME); Myristyltrimethylammonium bromide; 3-[(3-cholamidopropyl)dimethylammonio]-1-propanesulfonate (CHAPS); Tri-methyl-tetradecyammonium chloride (TTAC); chaotropic salts, such as guanidine hydrochloride, guanidinium thiocyanate, sodium thiocyanate, sodium iodide, sodium perchlorate, (tris(2-carboxyethyl)phosphine (TCEP) and urea. The amount of denaturing agent may be from about 0.2 to about 65 mM of the formulation.

The mouth's ecology and bacteria comprises over 700 species of bacteria, representing 30-60% of total saliva DNA with about 100 million microbes per 1 ml of saliva (Yang F, Ning K, Zeng X, Zhou Q, Su X, Yuan X. Characterization of saliva microbiota's functional feature based on metagenomic sequencing. SpringerPlus. 2016; 5(1):2098. doi:10.1186/s40064-016-3728-6.; Kilian, M., Chapple, I. L. C., Hannig, M., Marsh, P. D., Meuric, V., Pedersen, A. M. L., Tonetti, M. S., Wade, W. G., Zaura, E., The oral microbiome—an update for oral healthcare professionals, B r Dent J, 2016/1/18/print, 221 10 657 666, Nature Publishing Group http://dx.doi.org/10.1038/sj.bdj.2016.865.) Some of these bacteria have deleterious effects on DNA. The stabilizing formulation described herein therefore further comprises a biocide. Exemplary biocides for use within the formulations described herein include, but are not limited to, 5-chloro-2-methyl-4-isothiazolin-3-one (range ˜1% to 3%), sodium azide, 2-methyl-4-isothiazolin-3-one (range ˜0.25-1%), thimerosal, hypochlorite, and mixtures thereof. The amount of biocide and/or preservative in the composition can be from about 0.1% to about 2.5% of the formulation.

The chemical backbone and the purine bases of DNA are most stable at slightly alkaline pH, with an optimal stability generally recognized as being within a pH range of about 7-11, and desirably a pH of about 8. Below a pH of about 6, depurination (i.e., spontaneous loss of purine bases from the dedxyribose-phosphate backbone) can occur. Above a pH of about 10, spontaneous loss of amino groups from cytosine nucleotides may occur, thereby converting cytosine to uracil. Above a pH of about 12, DNA is denatured, converting it from the double-strand form to the single-strand form.

Reagents indicating sufficient mixing of the saliva and the stabilizing formation may be included as well as reagents that indicate contamination or degradation of the sample. In some embodiments, the stabilizing formulation described herein may include one, two, three, four, five, six, seven, eight, nine, ten or more detectable indicators. Detectable indicators include compositions that permit detection or similar determination of any detectable parameter that directly relates to a condition, process, pathway, induction, activation, inhibition, regulation, dynamic structure, state, contamination, degradation or other activity or functional or structural change in a biological sample, including but not limited to altered enzymatic or other biochemical or biophysical activity in the biological sample, and also including interactions between intermediates that may be formed as the result of such activities. A wide variety of detectable indicators are known to the art and can be selected for inclusion in the presently disclosed compositions and methods depending on the particular parameter or parameters that may be of interest for particular biological samples in particular sample storage applications. Non-limiting examples of parameters that may be detected by such detectable indicators include detection of the presence of one or more of an amine, an alcohol, an aldehyde, a thiol, a sulfide, a nitrite, avidin, biotin, an immunoglobulin, an oligosaccharide, a nucleic acid, a polypeptide, an enzyme, a cytoskeletal protein, a reactive oxygen species, a metal ion, pH, Na⁺, K⁺, CI⁻, a cyanide, a phosphate, selenium, a protease, a nuclease, a kinase, a phosphatase, a glycosidase, and a microbial contaminant, and others.

In some embodiments, such reagents may provide a visual color change in the sample upon mixing. Such colorimetric reagents are known to those skilled in the art and are chosen so that they do not interfere with the stabilization of the DNA in the saliva sample. Examples of a broad range of detectable indicators (including colorimetric indicators) that may be selected for specific purposes are described in Haugland, 2002 Handbook of Fluorescent Probes and Research Products—Ninth Ed., Molecular Probes, Eugene, Oreg.; Mohr, 1999 J. Mater. Chem., 9: 2259-2264; Suslick et al, 2004 Tetrahedron 60:1 1 133-1 1 138; Handbook of Fluorescent Dyes and Probes-1^{st} ed., R. W. Sabnis, Wiley) A detectable indicator may be a fluorescent indicator, a luminescent indicator, a phosphorescent indicator, a radiometric indicator, a dye, an enzyme, a substrate of an enzyme, an energy transfer molecule, or an affinity label among others.

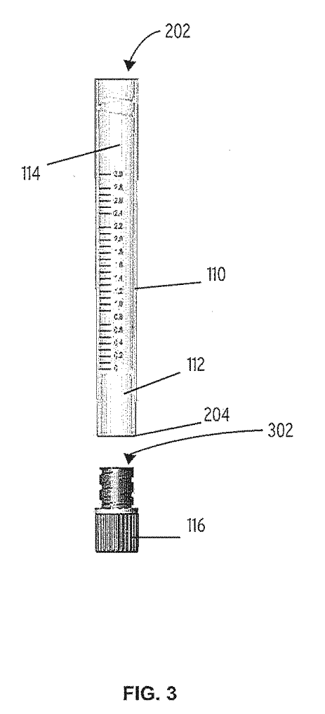

Appropriate amounts of the chelator, denaturing agent and biocide are combined in liquid form. In some embodiments, trehalose or another sugar may then be added to the mixture. An appropriate amount of the mixture is then allocated into a storage container such as, but not limited to, parts of a collection tube such as the cap for a collection tube. The liquid is then dried by any means generally used in order to create a semi-solid composition. In some embodiments, the solution is dried for about two hours at about 50 C in other embodiment. In other embodiments, it may be dried for up to about 4 hours, about 6 hours, about 7 hours, about 8 hours, about 9 hours, about 10 hours, about 12 hours or any fraction thereof. In some embodiments it is put in the cap of a collection tube such as that described in U.S. patent application Ser. No. 15/360,701 and that seen in FIG. 3.

The gel may be stored separately to be directly inserted into a saliva sample, or may be stored or otherwise contained in the sealing cap or other part of a saliva collection tube. In some embodiments, the gel may be covered by a seal and require puncturing to access. In other embodiments, it may be protected by a saliva-dissolvable membrane. In additional embodiments it may be placed in a sealing cap of a collection tube, such that when the collection tube is scaled the tube and manually agitated, the stabilizing formulation is put in contact with the collected saliva, rehydrating the stabilizing formulation and allowing for the stabilization of the DNA in the saliva sample.

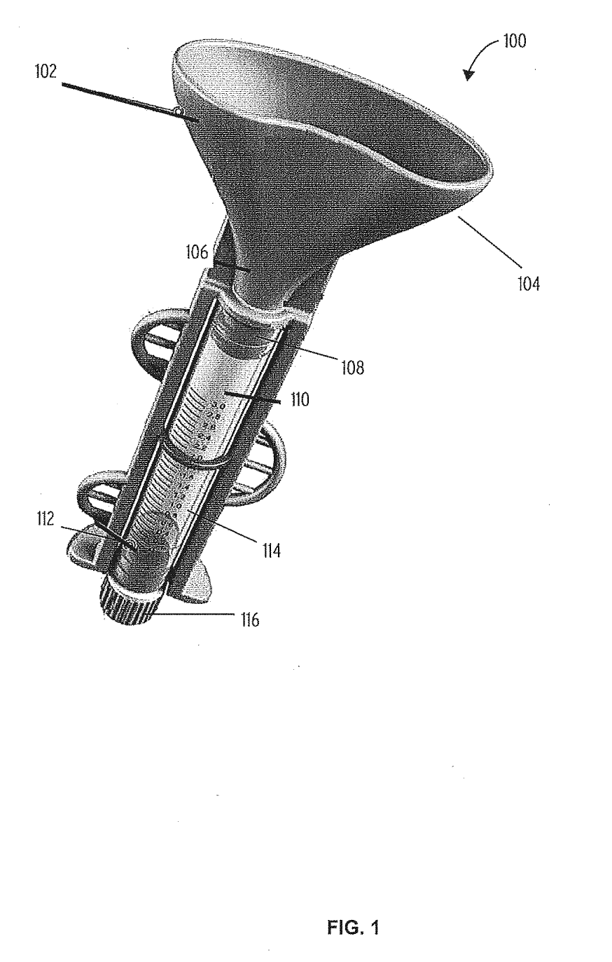

In some embodiments, the semi-solid gel may be stored in the cap of a collection device such as that described in U.S. patent application Ser. No. 15/360,701. Turning to FIG. 1, an exemplary sample collection device 110 comprising the semi-solid stabilizing formulation is shown. The sample collection device 100 includes a collection funnel 102 having an expanded open portion 104 adapted to fit comfortably against a patient's lower lip and a narrow portion 106 extending therefrom, the narrow portion 106 including a coupling 108 to receive a standardized collection tube 110 and thereby retain it at a fixed position relative to the funnel 102.

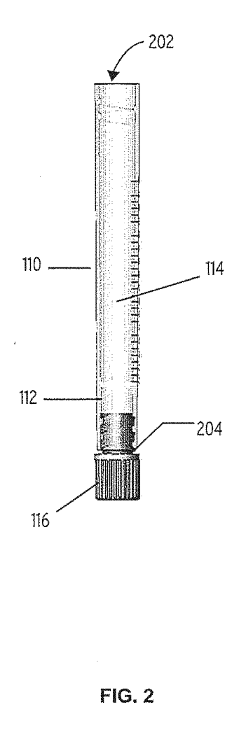

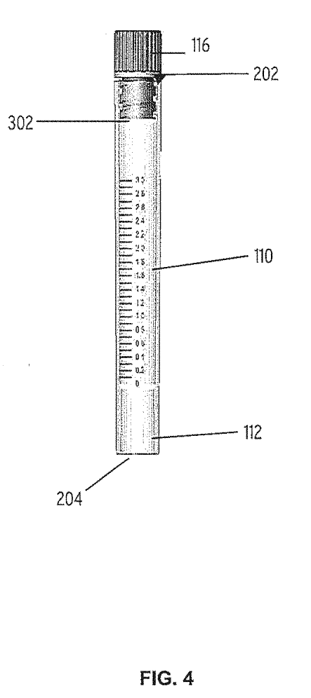

As seen in FIGS. 2-4, in one embodiment, collection tube 110 includes a first region with a collection portion 114 and a first open end 202 having a female threaded coupling and extends longitudinally to a second open end 204, and includes a base cavity 112 distinct from the collection portion 114 of the collection tube 110 proximate to the second open end 204 of the collection tube 110 which retains sealing element 116 when element 116 is not inserted in first end 202. Sealing element 116 has an open end 302 into which the semi-solid gel formulation can be stored. By storing the sealing element in the base cavity 112, its contents are protected from loss and requirements for additional steps for adding the buffer are eliminated. The sealing cap 116 is detached from the collection tube 110 and inserted in the first opening 202 of the collection tube 110 once the sample has been collected, sealing the saliva sample in the collection tube 110. The collection tube 110 is then agitated, putting the semi-solid stabilizing formulation in the open end of the sealing cap 302 in contact with the saliva sample, rehydrating the semi-solid stabilizing formulation, mixing it with the saliva sample and creating a stabilized saliva formulation which can be stored and transported at room temperature until processing.

The stability of the saliva/stabilizer formulation mixture was tested using real-time stability tests and accelerated stability tests as described in the examples below. Stability in this context refers to the presence of at least 50% of the initial high molecular weight DNA after storage at room temperature as determined by quantitation of the recovered gel band intensity by image analysis.

During real-time stability testing, the mixture was stored at recommended storage conditions (ambient) and monitored until it failed the specification. In accelerated stability tests, the product was stored at elevated stress conditions (such as temperature, humidity, and pH). Degradation at the recommended storage conditions was predicted using known relationships between the acceleration factor and the degradation rate.

Calculation of accelerated degradation due to temperature was determined using the Arrhenius equation:

k=Ae^{-E}^{_{α}}^{/(RT)}

where k is the rate constant; T is the absolute temperature (in ° Kelvin); A is the pre-exponential factor, a constant for each chemical reaction that defines the rate due to frequency of collisions in the correct orientation; Eₐ is the activation energy for the reaction (in Joules mol⁻¹) and R is the universal gas constant.

In other embodiments, stability testing may be accelerated through changes in humidity and pH. A model for parameter estimation and prediction of shelf life when temperature and pH are used as acceleration factors is given by Some et al. (Some I, et al. Stability parameter estimation at ambient temperature from studies at elevated temperatures. J Pharm Sci 2001; 90:1759-1766.)

The stabilizing formulation described herein will stabilize DNA in saliva for more than a week, preferably up to at least a month or more. In some embodiments, the DNA in saliva may be substantially stabilized by mixing the saliva with the stabilizing composition described herein and maintaining the mixture without refrigeration for a time period of at least 7, 8, 9, 10, 11, 12, 13, 14, 15, 16, 17, 18, 19, 20, 21 or more days. In further embodiments, the DNA in saliva will be stabilized for at least 4, 5, 6, 7, 8, 9, 10, 11, 12, 13, 14, 15, 16, 17, 18, 19, 20, 21, 22, 23, 24, 25, 26 or more weeks, or at least 6, 7, 8, 9, 10, 11, 12, 13, 14, 15, 16, 17, 18 or more months, or at least 2, 3, 4, 5 or more years.

When desired, the samples may be processed or analyzed for nucleic acid molecules contained therein by isolating such biomolecules from other components of the sample while at the same time separating them from the storage composition, which does not compromise the biological activity of the nucleic acid.

In some embodiments, the semi-solid formulation described herein prevents degradation of at least 50%, at least 60%, at least 70%, at least 80%, at least 90% of recoverable DNA in the mixture, relative to an amount of DNA that is recoverable from the sample when stored for the same time period at −20° C. without the stabilizing formulation described herein. In some embodiments, the stabilizing formulation described herein substantially prevents degradation of at least 50%, 75%, 80%, 85%, 90%, 95% or more of the recoverable DNA in the mixture, relative to an amount of DNA that is recoverable from the sample when stored for the same time period at less than −20° C., −30° C., −40° C., −50° C., −60° C., −70° C., or −80° C. without the herein described composition for substantially stable biological sample storage.

It should be understood that in addition to the ingredients, particularly mentioned above, the formulations of the present invention may include other agents conventional in the art having regard to the type of formulation in question.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The above disclosure generally describes the present invention. A more complete understanding can be obtained by reference to the following examples. These examples are described solely for purposes of illustration and are not intended to limit the scope of the invention. Although specific terms have been employed herein, such terms are intended in a descriptive sense and not for purposes of limitations.

### EXAMPLES

### Example 1

### Method of Saliva Collection

Saliva was collected using an unstimulated saliva collection method in which 5 minutes prior to collection of unstimulated saliva samples, individuals were asked to rinse orally with water and discard the water. Study members were then asked to spit into a collection device until about 2.0 mL had been collected.

### Example 2

### Method of Making Semi-Solid Gel

30 mL of 500 mM EDTA, pH=8.0, 5 mLs of 20% SDS, 500 μl of ProClin 300 (Sigma-Aldrich, St. Louis, Mo.) and 6 g trehalose were mixed. After mixing, 100 μl of the resulting solution was aliquoted and dried for 2 hours at 50 C to form a semi-solid DNA preserving gel formulation.

### Example 3

### DNA Preservation

The gel described in Example 2 was placed in the cap of a collection device described in U.S. patent application Ser. No. 15/26,071, Whole Saliva Sample Collection Device and Method filed Nov. 23, 2016. Saliva was expectorated into the collection tube through the removable mouthpiece until 2.0 mL has been collected. Once sample collection was complete, the tube and cap assembly as described in U.S. patent application Ser. No. 15/26,071, Whole Saliva Sample Collection Device and Method filed Nov. 23, 2016, was removed from the plastic external housing by unscrewing. The cap was then removed from the bottom of the tube and placed onto the top of the collection tube and screwed down tightly to secure the specimen. The sealed collection tube was then inverted/shaken for 1 minute to mix the sample with the gel embedded into the cap, providing a stabilized sample of salivary DNA which was then stored at room temperature.

### Example 4

### Stabilization of DNA in Saliva Sample and Analysis of DNA Preservation

To form the semi-solid gel formulation, 30 mL of 500 mM EDTA, pH=8.0, 5 mLs of 20% SDS, 500 μl of ProClin 300 (Sigma-Aldrich, St. Louis, Mo.) and 6 g trehalose were mixed. 100 μl was then aliquoted and dried for 2 hours at 50 C to form a semi-solid gel formulation. Saliva was collected from 10 individuals using an unstimulated collection method in which 5 minutes prior to collection of unstimulated saliva samples, ten individuals were asked to rinse orally with water and discard the water. The study members were then asked to spit into a collection tube until 2.0 mL had been collected. These samples were combined to form a single pool. This pool was then split between controls and formulation tubes.

Duplicate samples were placed at 37° C. for the accelerated stability study (samples at 37 C for 5 days=˜equivalent to 15 days RT, for 12 days=˜equivalent to 32 days RT, 20 days=˜equivalent to 56 days RT).

Aliquots were then tested for DNase activity using DNaseAlert™ QC System (Thermo-Fisher Scientific, Boston, Mass.) which detects DNaso I, Benzonasc, Exonuclease III, mung bean nuclease, micrococcal nuclease, Bal31 nuclease, S1 nuclease and T7 endonuclease. 90 microliters of raw saliva, saliva mixed with the semi-solid gel formulation, or saliva mixed with trehalose and proclin300 only were then added to each well in a 96 well plate. To perform the analysis, 10 μl of re-suspended DNase Alert substrate was added to each well. No buffer was used. Time point readings were taken at t=0, and then periodically out until at least 50 hours

DNA was using Zymo Quick-DNA Universal Kit #D4068 (Zymo Research, Irvine Calif.). Prior to loading into a well in a gel, 200 μl of the samples were placed in a microcentrifuge tube to which was added 200 μl BioFluid & Cell Buffer (Red) and 20 μl Proteinase K. The mixture was then mixed thoroughly and then incubated at 55° C. for 10 minutes. After incubation, 1 volume of Genomic Binding Buffer was added to the digested sample. The sample was then again mixed thoroughly. The mixture was then transferred to a Zymo-Spin™ IIC-XL Column in a Collection Tube and centrifuged at ≥12,000×g for 1 minute. The flow through was subsequently discarded. 400 μl DNA Pre-Wash Buffer was then added to the spin column in a new Collection Tube and centrifuged at ≥12,000×g for 1 minute and the collection tube was emptied. 700 μl g-DNA Wash Buffer was added to the spin column which was then centrifuged at ≥12,000×g for 1 minute. The collection tube was then emptied. 200 μl g-DNA Wash Buffer was then added to the spin column which was then centrifuged at ≥12,000×g for 1 minute. The collection tube was then discarded with the flow through. The spin column was then transferred to a clean microcentrifuge tube. ≥50 μl DNA Elution Buffer was added directly to the matrix which was then incubated for 5 minutes at room temperature, then centrifuged at maximum speed for 1 minute to elute the DNA. To increase yields, eluted DNA in buffer was then reloaded onto the matrix for 5 minutes at room temperature and re-centrifuged. 10 μl of the isolated DNA from each sample was then loaded onto a 1.2% Sybr Safe Gel from Invitrogen (Carlsbad, Calif.). Lambda DNA HindIII marker (ThermoFisher Scientific, Boston, Mass.) and Invitrogen E-gel 96 High Range DNA marker, ref #12352-019 (ThermoFisher Scientific, Boston, Mass.) were used as molecular-weight size markers for comparison to determine the size of the bands in the gel. Lambda HindIII DNA Markers provide a set of double-stranded DNA size standards, 23130 bp, 9416 bp, 6557 bp, 4361 bp, 2322 bp, 2027 bp, 564 bp and 125 bp in length.

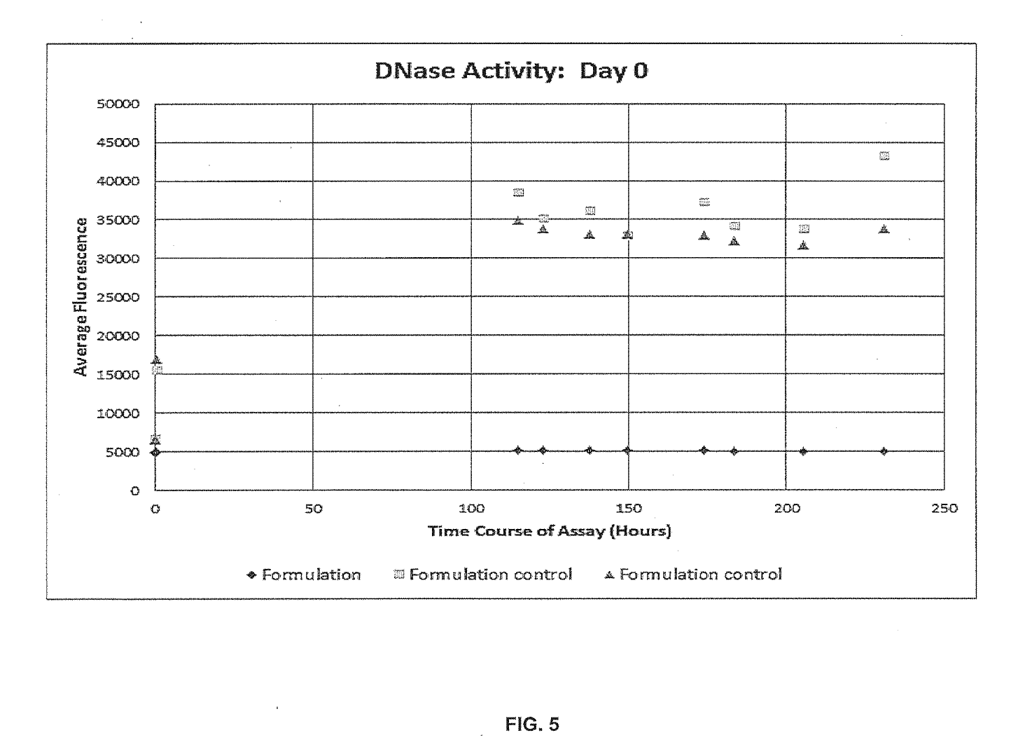

As measured by average fluorescence 540/590 Gain 130 and shown in Table 1 and FIG. 5, from time 0 to 231 hours the DNase activity in saliva combined with the semi-solid gel formulation displayed no increase in DNase activity, indicating substantial preservation of the DNA structure in saliva.

TABLE 1
DNase Assay from Day O

| | | | | | | | | | | | | % activity |
|---|---|---|---|---|---|---|---|---|---|---|---|---|
| | | | | | | | | | | | | compared to |
| | | Hours | | | | | | | | | | time = 0 |
| | | 0 | 0.5 | 115 | 123 | 138 | 150 | 174 | 184 | 206 | 231 | T231/T0 |
| Day 0 | Formulation | 4951 | 4976 | 5202 | 5143 | 5243 | 5141 | 5108 | 4993 | 5007 | 4976 | 101% |
| | Formulation | 6647 | 15653 | 38505 | 35217 | 36168 | 33007 | 37245 | 34201 | 33810 | 43292 | 651% |
| | control | | | | | | | | | | | |
| | Raw | 6615 | 16976 | 34959 | 33819 | 33122 | 33116 | 32936 | 32235 | 31756 | 33761 | 510% |
| | saliva | | | | | | | | | | | |

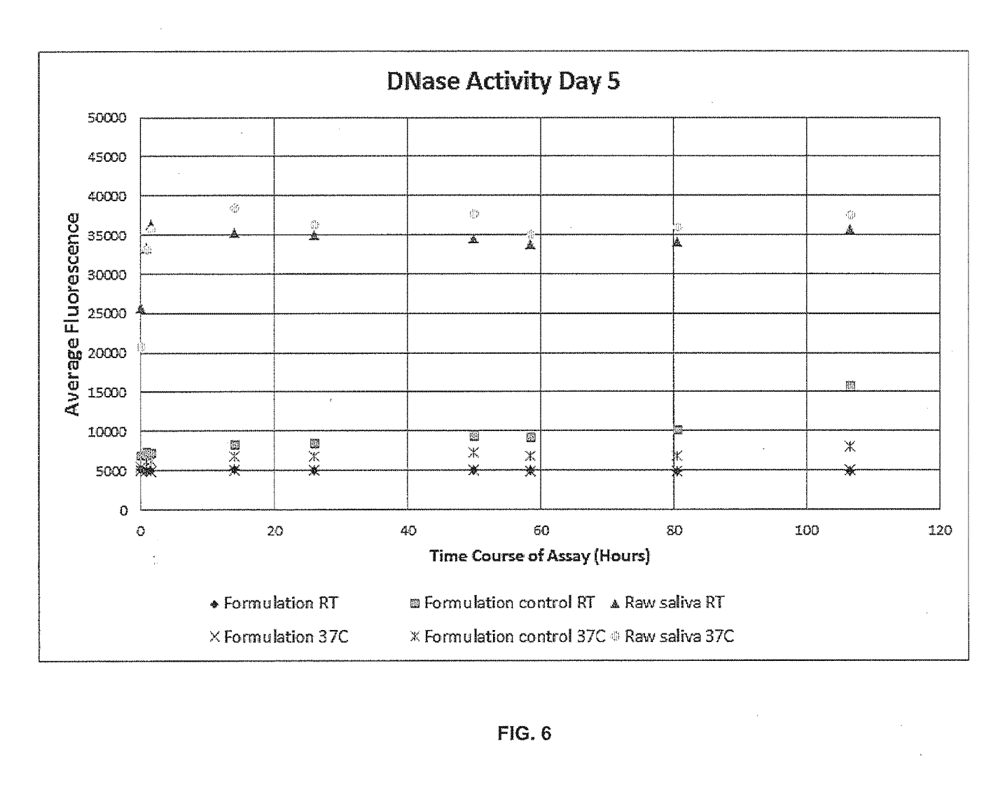

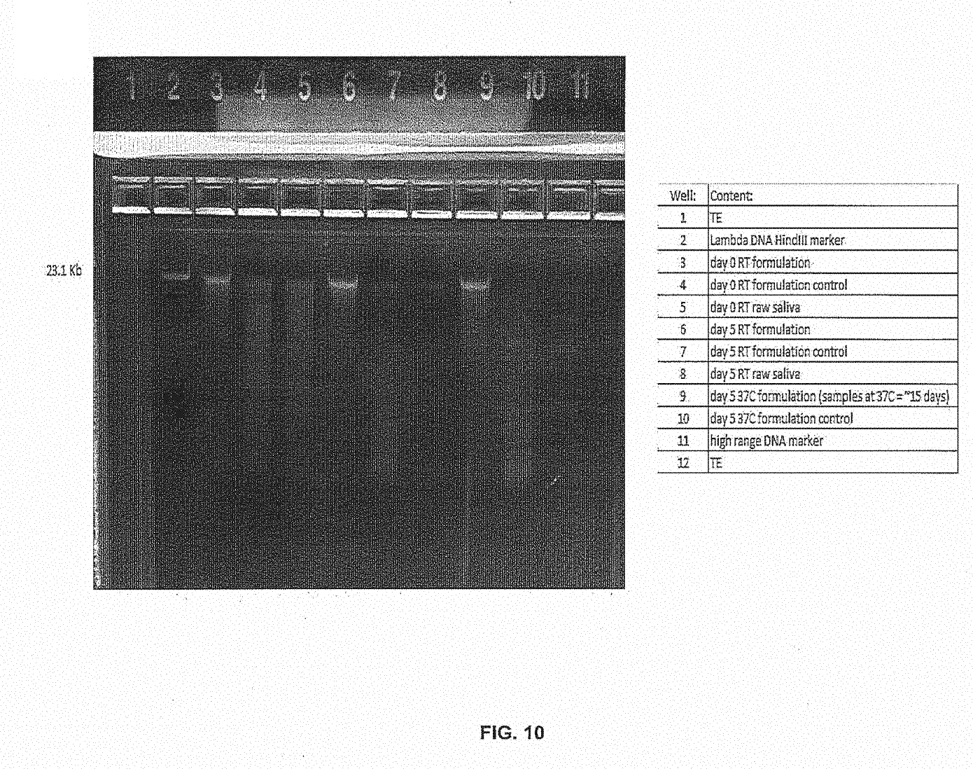

This stability continued through Day 5 shown in Table 2 and FIG. 6. The preservation of the DNA structure is additionally evident in FIG. 10 which clearly shows a band slightly below 23.1 kb on day 5 of saliva mixed with the formulation, whereas the band is absent in the formulation control. Furthermore, the stability was not affected by increased temperature (37 C). The Arrhenius equation states that 5 days at 37 C was equivalent to 15 days in real time at room temperature. DNase activity in the saliva/semi-solid gel formulation for 5 days at room temperature or 37 C was substantially unchanged.

TABLE 2
DNase Assay starting on Day 5

| | | | | | | | | | | | % activity |
|---|---|---|---|---|---|---|---|---|---|---|---|
| | | | | | | | | | | | compared to |
| | | Hours | | | | | | | | | time = 0 |
| | | 0 | 0.75 | 1.5 | 14 | 26 | 50 | 58.5 | 80.5 | 106.5 | T106.5/T0 |
| Day 5 | Formulation | 5099 | 4995 | 5062 | 5101 | 5080 | 5028 | 4975 | 4891 | 4880 | 96% |
| | RT | | | | | | | | | | |
| | Formulation | 6987 | 7397 | 7367 | 8404 | 8552 | 9444 | 9218 | 10113 | 15891 | 227% |
| | control RT | | | | | | | | | | |
| | Raw | 25794 | 33346 | 36311 | 35356 | 35060 | 34573 | 33856 | 34077 | 35800 | 139% |
| | saliva | | | | | | | | | | |
| | RT¹ | | | | | | | | | | |
| | Formulation | 5034 | 4973 | 4917 | 5065 | 5062 | 4978 | 4847 | 4816 | 4959 | 99% |
| | 37 C. | | | | | | | | | | |
| | Formulation | 5472 | 6087 | 6273 | 6804 | 6876 | 7360 | 6775 | 6778 | 8065 | 147% |
| | control 37 C. | | | | | | | | | | |
| | Raw | 20785 | 33187 | 35792 | 38574 | 36396 | 37774 | 35151 | 36079 | 37562 | 181% |
| | saliva 37 C. | | | | | | | | | | |

¹Assay substrate is substantially hydrolyzed from start of assay

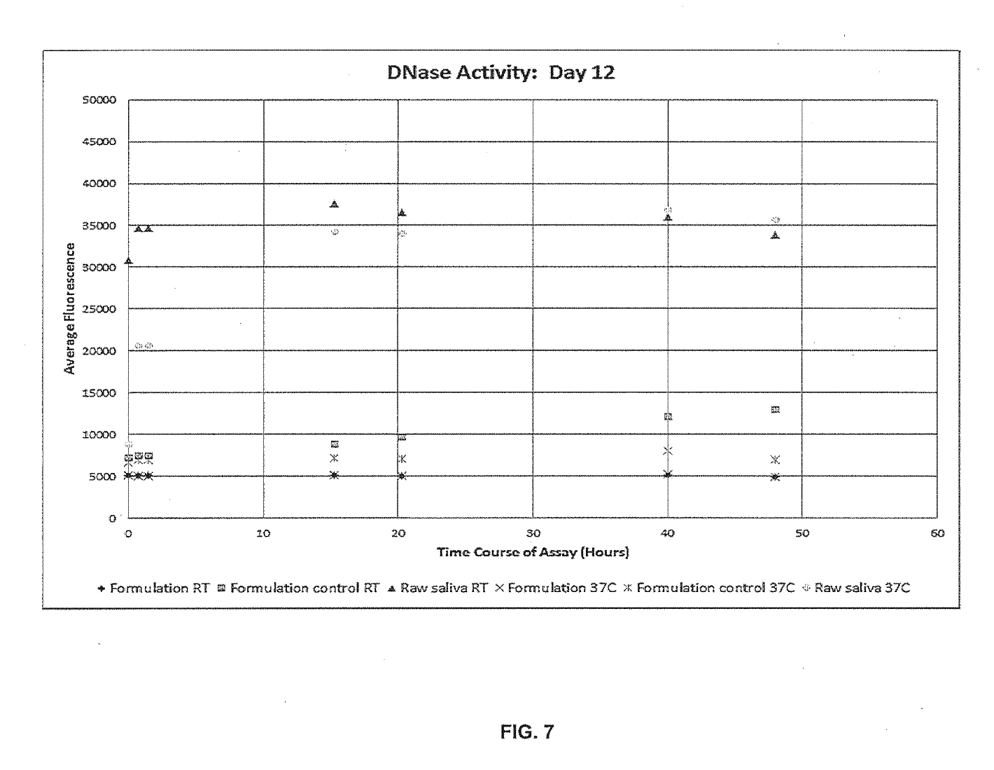

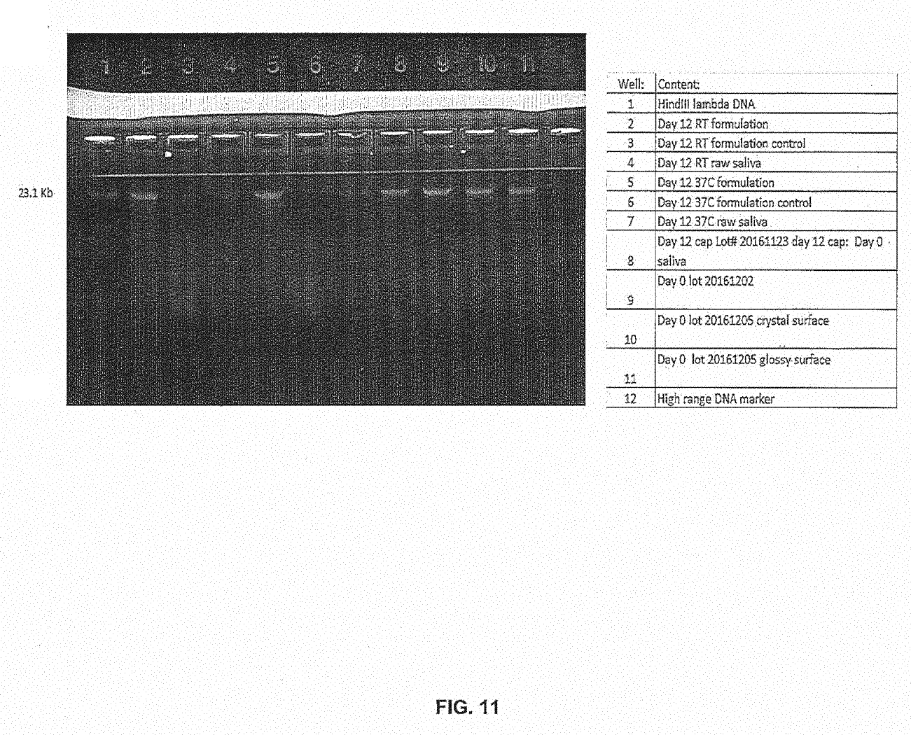

This stability continued through Day 12 at room temperature as shown in Table 3 and FIG. 7 with the DNase activity in various lots of the semi-solid formulation in combination with saliva demonstrating substantially unchanged DNase activity. DNA preservation is visible as a band slightly lower than 23.1 kb in the gel in FIG. 11 where there is a clear band at day 12 of the saliva combined with the formulation and no band on day 12 for saliva mixed with the formulation control.

TABLE 3
DNase Assay starting on Day 12

| | | | | | | | | | % activity |
|---|---|---|---|---|---|---|---|---|---|
| | | | | | | | | | compared to |
| | | Hours | | | | | | | time = 0 |
| | | 0 | 0.75 | 1.5 | 15.25 | 20.25 | 40 | 48 | T48/T0 |
| Day 12 | Formulation RT | 5173 | 5149 | 5149 | 5085 | 5025 | 5115 | 4960 | 96% |
| | Formulation | 7284 | 7460 | 7460 | 8857 | 9743 | 12184 | 12995 | 178% |
| | control RT | | | | | | | | |
| | Raw saliva RT | 30927 | 34695 | 34695 | 37622 | 36529 | 36044 | 33869 | 110% |
| | Formulation 37 C. | 5124 | 5027 | 5027 | 5144 | 5017 | 5208 | 4963 | 97% |
| | Formulation | 6652 | 6977 | 6977 | 7176 | 7091 | 7998 | 6960 | 105% |
| | control 37 C. | | | | | | | | |
| | Raw saliva 37 C. | 8896 | 20707 | 20707 | 34329 | 34124 | 36824 | 35685 | 401% |

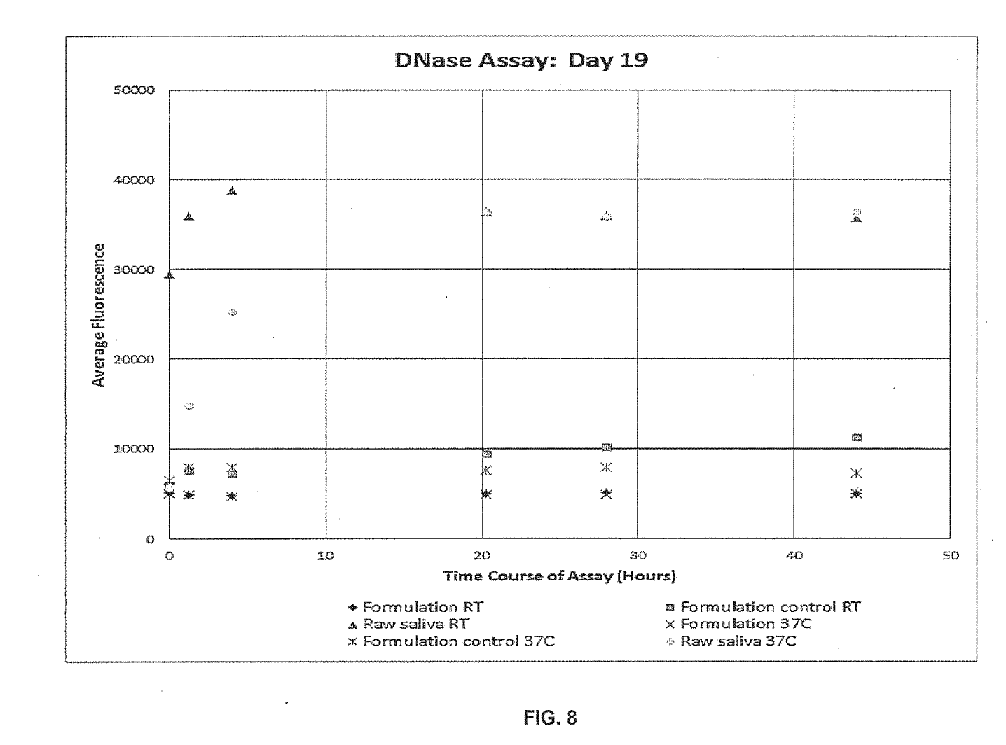

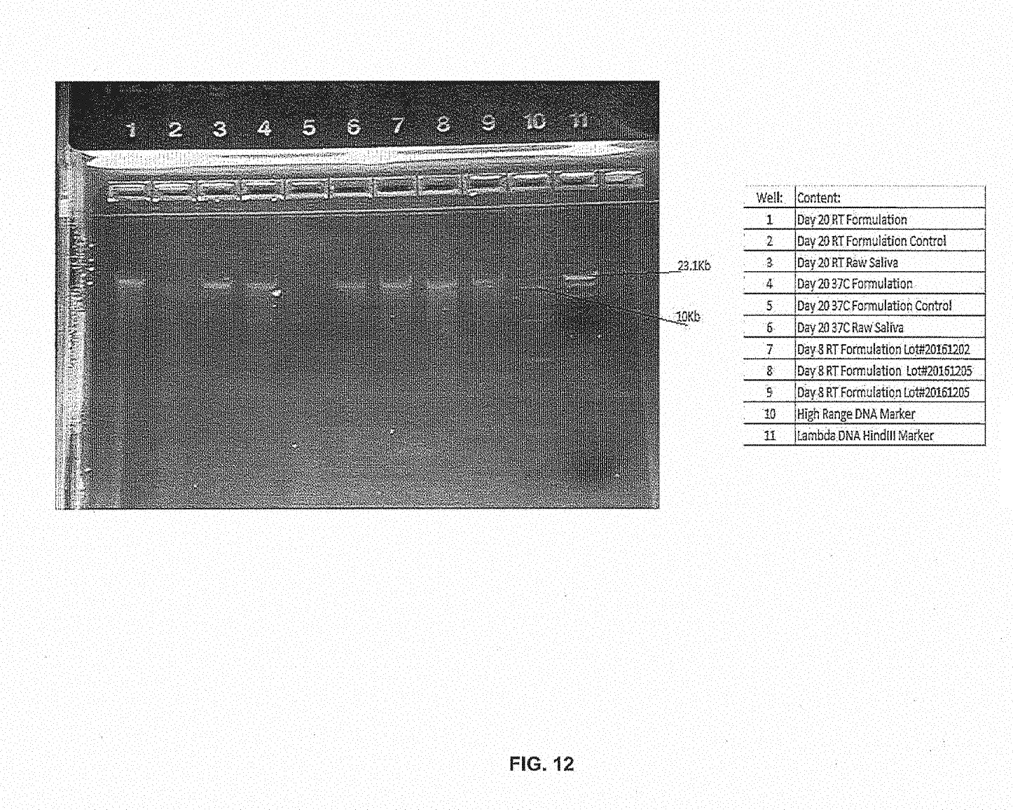

As shown in Table 4 and FIG. 8, the DNase activity of the combined saliva and formulation continued to show stability with little change in the DNase activity through Day 19. Significant amounts of DNA at day 20 are visible in FIG. 12, where there is a band slightly below 23.1 kb in saliva mixed with the formulation as seen in lane 1, but not in the mixture of saliva with the control as seen in lane 2.

TABLE 4
DNase Assay starting on Day 19

| | | | | | | | | % activity |
|---|---|---|---|---|---|---|---|---|
| | | | | | | | | compared to |
| | | Hours | | | | | | time = 0 |
| | | 0 | 1.25 | 4 | 20.25 | 28 | 44 | T44/T0 |
| Day 19 | Formulation RT | 5034 | 4897 | 4708 | 5004 | 5121 | 4981 | 99% |
| | Formulation control RT | 5971 | 7599 | 7227 | 9418 | 10202 | 11280 | 189% |
| | Raw saliva RT² | 29507 | 35948 | 38769 | 36451 | 36003 | 35809 | 121% |
| | Formulation 37 C. | 4934 | 4833 | 4621 | 4915 | 4905 | 4975 | 101% |
| | Formulation control 37 C. | 6453 | 7919 | 7868 | 7541 | 7882 | 7267 | 113% |
| | Raw saliva 37 C. | 5718 | 14827 | 25252 | 36364 | 35914 | 36457 | 638% |

²Assay substrate is substantially hydrolyzed from start of assay

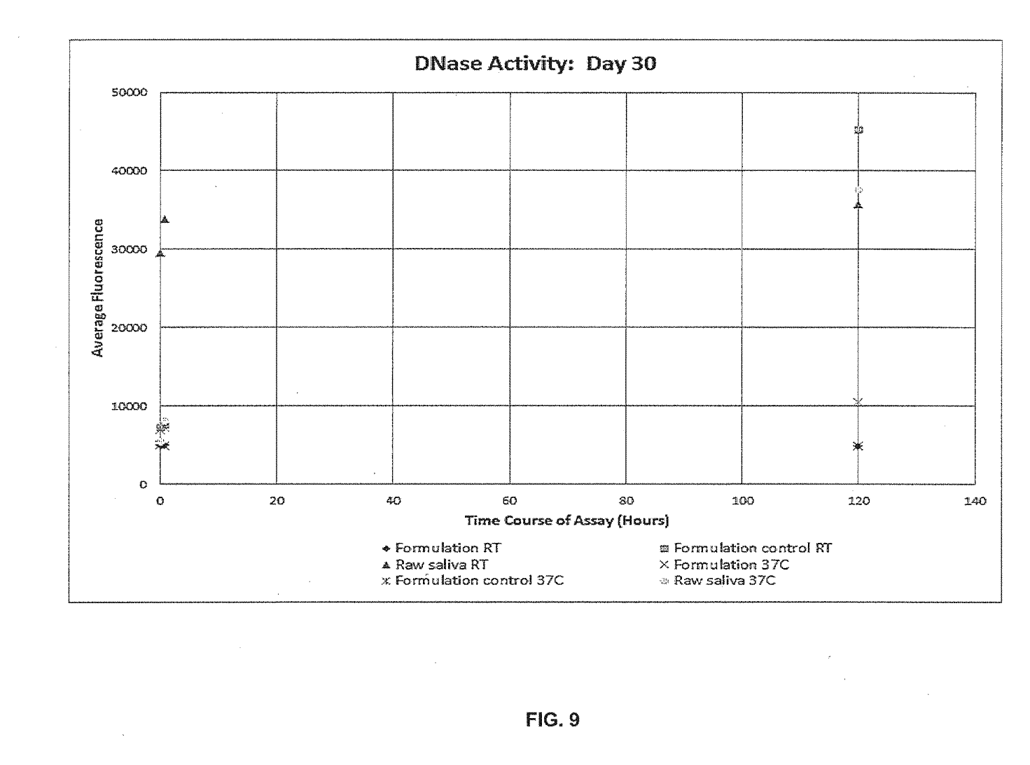

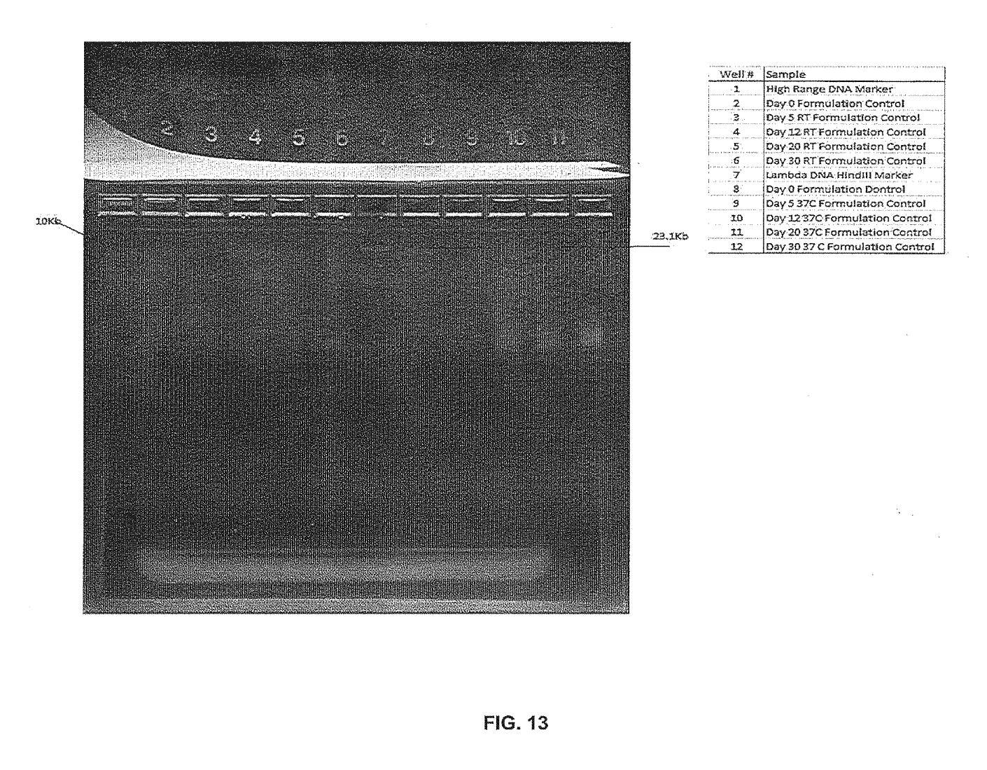

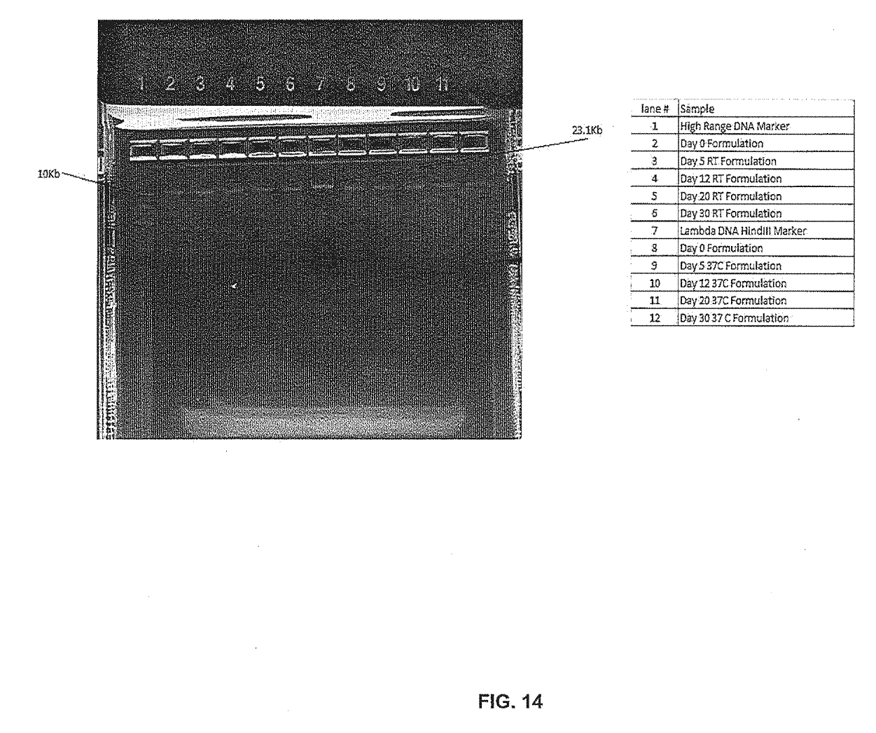

As shown in Table 5 and FIG. 9, the DNase activity of the combined saliva and formulation continued to show stability with little change in the DNase activity through Day 30. Significant amounts of DNA at day 30 are visible in FIG. 14, where there is a band slightly below 23.1 kb in saliva mixed with the formulation as seen in lane 6, but not in the mixture of saliva with the control as seen in corresponding lane 6 of 30 days using the formulation control as seen in FIG. 13.

TABLE 5
DNase Assay starting on Day 30

| | | | | | % activity |
|---|---|---|---|---|---|
| | | | | | compared to |
| | | | | | time = 0 |
| | Hours | 0 | 0.75 | 120 | T120/T0 |
| Day 30 | Formulation | 5043 | 5048 | 4871 | 97% |
| | RT | | | | |
| | Formulation | 7316 | 7671 | 45332 | 620% |
| | control RT | | | | |
| | Raw saliva | 29609 | 33816 | 35777 | 121% |
| | RT³ | | | | |
| | Formulation | 4944 | 4880 | 4859 | 98% |
| | 37C | | | | |
| | Formulation | 6887 | 7294 | 10425 | 151% |
| | control 37C | | | | |
| | Raw saliva | 5416 | 8251 | 37711 | 696% |
| | 37C | | | | |

³Assay substrate is substantially hydrolyzed from start of assay

As shown in the gel in FIG. 13, there is significant DNA degradation in saliva mixed with the formulation control (trehalose and proclin300 only) at Day 0, Day 5, Day 12 and Day 30 as seen by an absence of a band slightly below 23.1 kb. In contrast, as shown in the gel in FIG. 14, when saliva is mixed with the DNA stabilizing formulation described herein, there is preservation of a band slightly below 23.1 kb at Day 0, Day 5, Day 12 and Day 30 as well as the accelerated studies at 37 C. Dried gel had the same anti-DNase activity when rehydrated with saliva as fresh solution mixed with saliva (See Columns 2 and 8-11 of FIG. 11 for comparison).

### Example 5

### Analysis of Biocidal Control

Raw saliva, saliva with EDTA/Trehalose/Proclin and saliva with EDTA/Trehalose/Proclin/l % SDS were tested for microbial content including total aerobic microbial count (TAMC) and total yeasts and molds count (TYMC) and total colony forming units were determined after 3 and 7 days respectively.

In order to measure microbial growth, pre-poured plates (USP qualified from Hardy Diagnostics Santa Maria, Springboro) were utilized. TSA Plates were used for TAMC (total aerobic microbial counts). Samples of saliva alone, saliva with EDTA/Trehalose/Proclin and saliva with EDTA/Trehalose/Proclin and 1% SDS were diluted 1:10 in peptone buffer and 100 μl was spread on the plates. Plates were incubated for 1-3 days at 37 C. Dextrose agar plates were used for TYMC (total yeast mold count) 100 μl of undiluted samples were plated. These plates were incubated for 5-7 days at 22 C and total colony forming units determined.

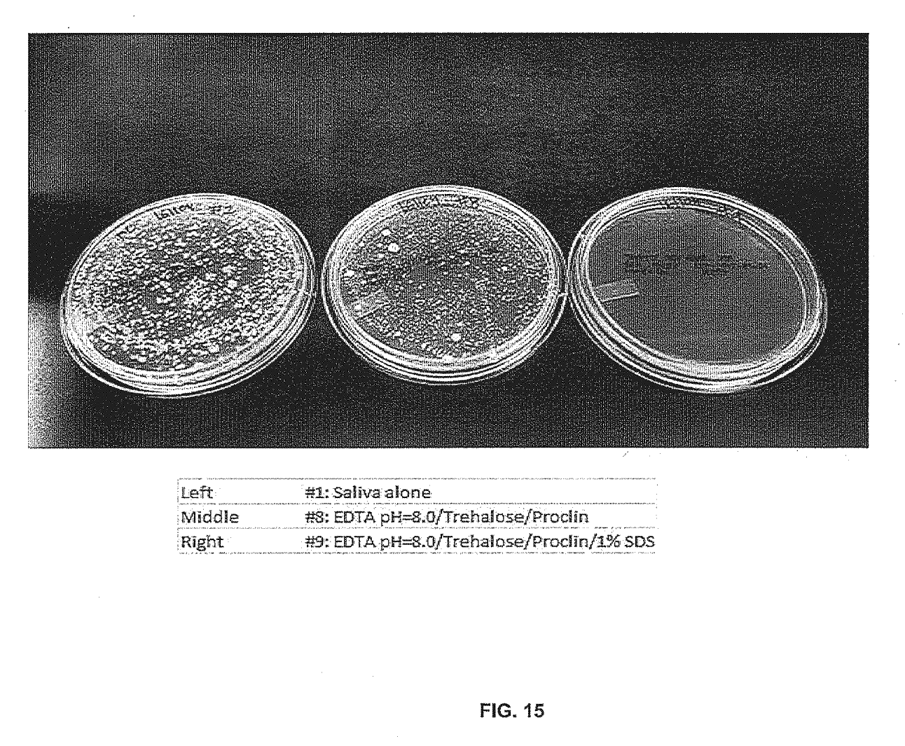

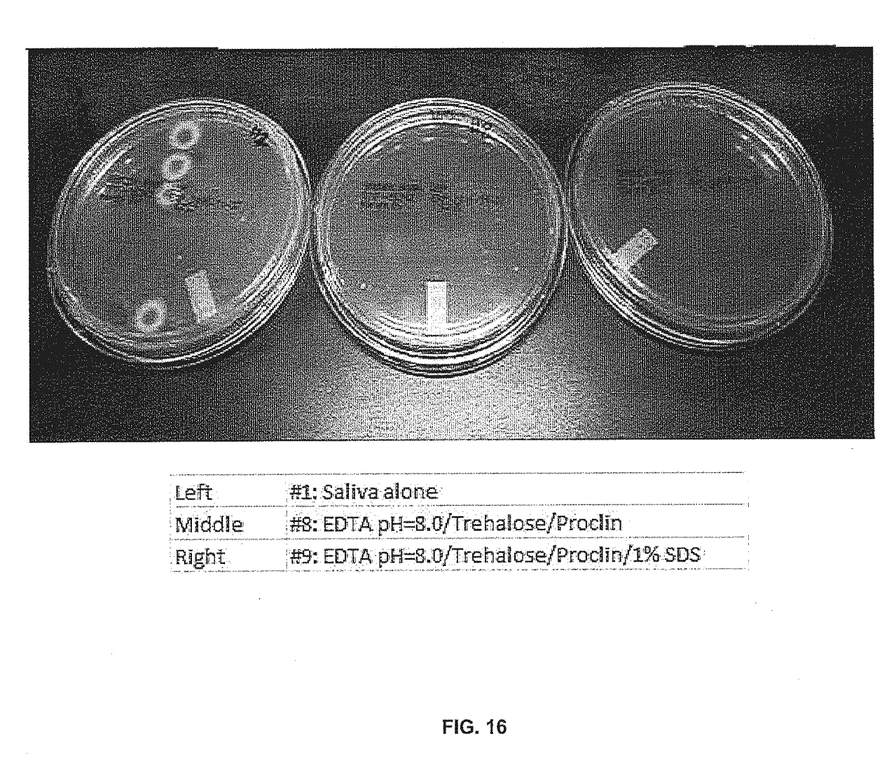

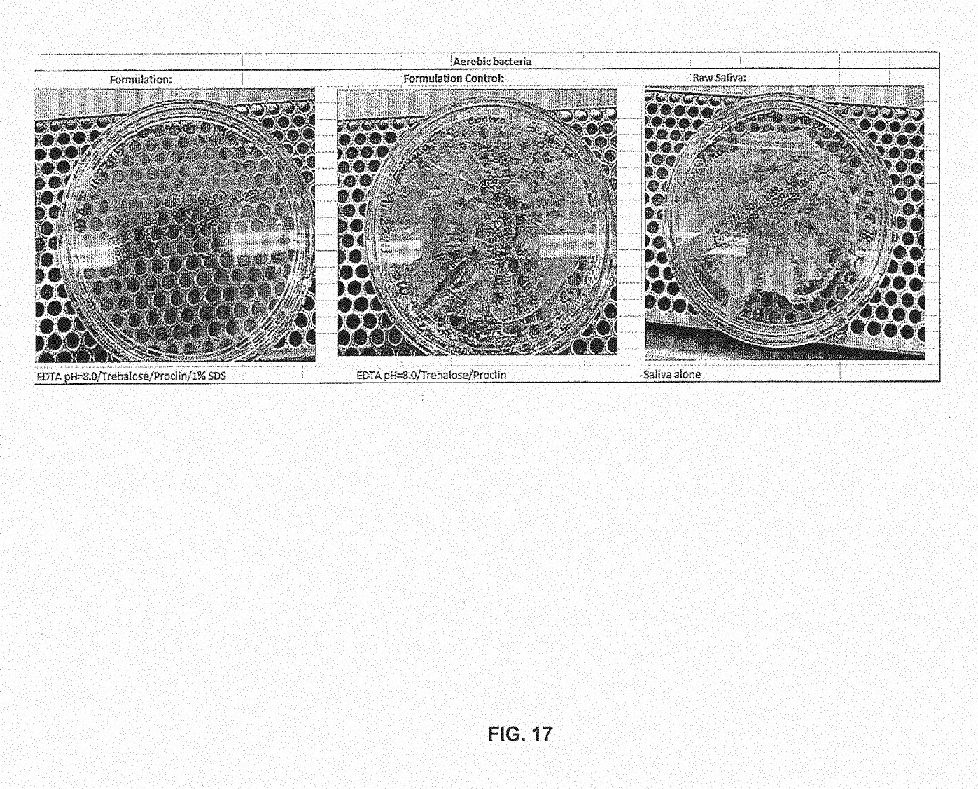

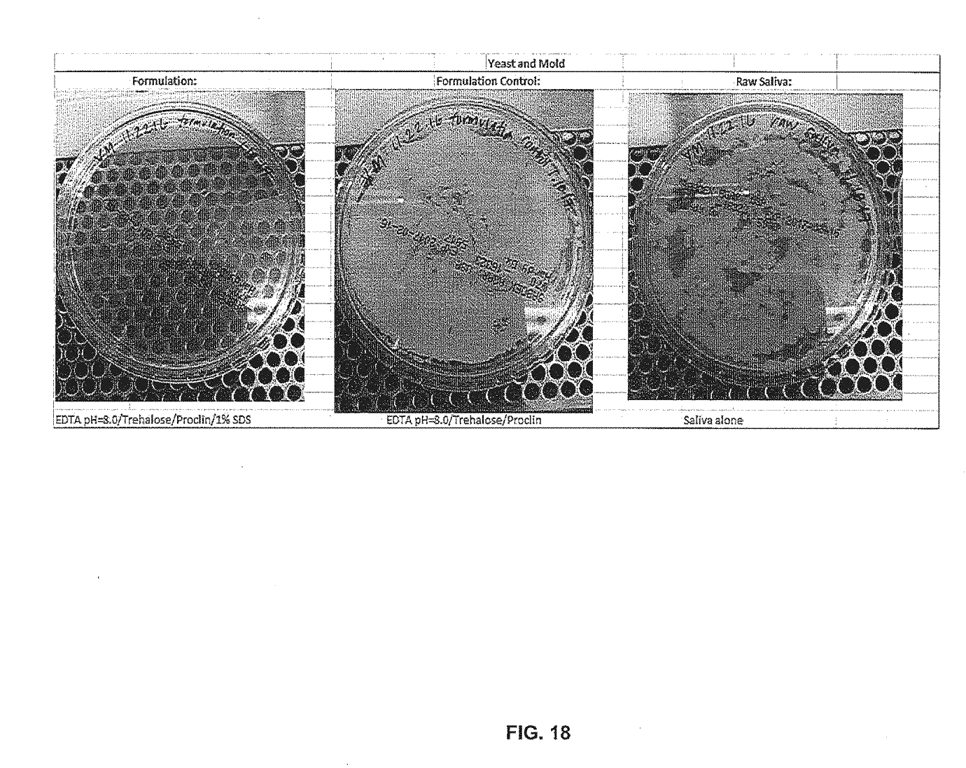

As shown in Tables 6 and 7, and FIGS. 15, 16, 17 and 18, the formulation of EDTA/Trehalose/Proclin and 1% SDS was capable of inhibiting bacterial, mold and yeast growth in saliva for at least 56 days from plating, creating a stabilized form of saliva suitable for storage and transport at room temperature.

TABLE 6
Total Aerobic Colony Counts after three days

| | | TAMC |
|---|---|---|
| Sample | Treatment concentration | 3-day check |
| Plate 1 | Saliva pool alone | 876 |
| Plate 8 | EDTA | 80 |
| | pH = 8.0/Trehalose/Proclin | |
| Plate 9 | EDTA | 0 |
| | pH = 8.0/Trehalose/Proclin/ | |
| | 1% SDS | |

TABLE 7
Total Yeast and Mold Colony Counts after 7 days

| | | TYMC |
|---|---|---|
| | | 7 day |
| Sample | Untreated | check |
| Plate 1 | Saliva pool alone | 7 |
| Plate 8 | EDTA | ~31 |
| | pH = 8.0/Trehalose/Proclin | |
| Plate 9 | EDTA | 0 |
| | pH = 8.0/Trehalose/Proclin/ | |
| | 1% SDS | |

What is claimed is:

1. A method for preserving deoxyribonucleic acid (DNA) in saliva comprising:
a) obtaining the saliva from a subject;
b) contacting the saliva with a semi-solid stabilizing composition comprising a chelating agent, a denaturing agent, a biocide and trehalose, to form a liquid mixture; and
wherein the liquid mixture stabilizes the DNA for at least 7 days at room temperature.

2. The method of claim 1, wherein the DNA is stable for more than 19 days at room temperature.

3. The method of claim 1, wherein the DNA is stable for more than 30 days at room temperature.

4. The method of claim 1, wherein the chelating agent is ethylene glycol-bis(β-aminoethyl ether)-N,N,N′,N′-tetraacetic acid (EGTA), ethylenediamine tetraacetic acid (EDTA), cyclohexane diaminetctraacetate (CDTA), diethylenetriamine pentaacetic acid (DTPA), tetraazacyclododecanetetraacetic acid (DOTA), tetraazacyclotetradecanetetraacetic acid (TETA), or desferrioximine.

5. The method of claim 4, wherein the chelating agent is EDTA.

6. The method of claim 1, wherein the denaturing agent is Tween, Triton, Nonidet, Igepal, Tergitol, sodium dodecyl sulfate (SDS), N-lauroyl sarcosine; diethyl glycol monoethyl ether (DGME), Myristyltrimethylammonium bromide, 3-[(3-cholamidopropyl) dimethylammonio]-1-propanesulfonate (CHAPS), Tri-methyl-tetradecyammonium chloride (TTAC), guanidine hydrochloride, guanidinium thiocyanate, sodium thiocyanate, sodium iodide, sodium perchlorate, TCEP (tris(2-carboxyethyl)phosphine or urea.

7. The method of claim 1, wherein the biocide is 5-chloro-2-methyl-4-isothiazolin-3-one, sodium azide, 2-methyl-4-isothiazolin-3-one, thimerosal, or hypochlorite.

8. A method for preserving deoxyribonucleic acid (DNA) in saliva comprising:
a) collecting a saliva sample in a first region of a collection tube with a first open end;
b) storing a semi-solid stabilizing composition comprising a chelating agent, a denaturing agent, a biocide and trehalose in an interior of a first end of a sealing element, wherein the first end of the sealing element is located in a second region of the collection tube with a second open end;
c) removing the scaling element from the second open end of the collection tube;
d) closing the collection tube by inserting the first end of the sealing element in the first open end of the collection tube; and
e) agitating the collection tube so that the saliva sample combines with the semi-solid stabilizing composition forming a liquid mixture,
wherein mixing of the semi-solid stabilizing composition and the saliva stabilizes the DNA in the saliva for at least 7 days at room temperature.

9. The method of claim 8, wherein the DNA is stabilized for at least 90 days.

10. The method of claim 8, wherein the chelating agent is ethylene glycol-bis(β-aminoethyl ether)-N,N,N′,N′-tetraacctic acid (EGTA), ethylenediamine tetraacetic acid (EDTA), cyclohexane diaminetetraacetate (CDTA), diethylenetriamine pentaacetic acid (DTPA), tetraazacyclododecanetetraacetic acid (DOTA), tetraszacyclotetradecanetetraacetic acid (TETA), or desferrioximine.

11. The method of claim 10, wherein the chelating agent is EDTA.

12. The method of claim 8, wherein the denaturing agent is Tween, Triton, Nonidet, Igepal, Tergitol, sodium dodecyl sulfate (SDS), N-lauroyl sarcosine; diethyl glycol monoethyl ether (DGME), Myristyltrimethylammonium bromide, 3-[(3-cholamidopropyl)dimethylammonio]-1-propanesulfonate (CHAPS), Tri-methyl-tetradecyammonium chloride (TTAC), guanidine hydrochloride, guanidinium thiocyanate, sodium thiocyanate, sodium iodide, sodium perchlorate, or urea.

13. The method of claim 8, wherein the biocide is 5-chloro-2-methyl-4-isothiazolin-3-one, sodium azide, 2-methyl-4-isothiazolin-3-one, thimerosal, or hypochlorite.

14. A saliva stabilizing formulation for stabilizing DNA in saliva comprising:
a denaturing agent, a chelating agent, a biocide and trehalose, wherein the saliva stabilizing formulation is a semi-solid gel.

15. The saliva stabilizing formulation of claim 14, wherein the chelating agent is ethylene glycol-bis(β-aminoethyl ether)-N,N,N′,N′-tetraacetic acid (EGTA), ethylenediamine tetraacetic acid (EDTA), cyclohexane diaminetetraacetate (CDTA), diethylenetriamine pentaacetic acid (DTPA), tetraazacyclododecanetetraacetic acid (DOTA), tetraazacyclotetradecanetetraacetic acid (TETA), or desferrioximine.

16. The saliva stabilizing formulation of claim 15, wherein the chelating agent is EDTA.

17. The saliva stabilizing formulation of claim 14, wherein the denaturing agent is Tween, Triton, Nonidct, Igepal, Tergitol, sodium dodecyl sulfate (SDS), N-lauroyl sarcosine; diethyl glycol monoethyl ether (DOME), Myristyltrimethylammonium bromide, 3-[(3-cholamidopropyl)dimethylammonio]-1-propanesulfonate (CHAPS), Tri-methyl-tetradecyammonium chloride (TTAC), guanidine hydrochloride, guanidinium thiocyanate, sodium thiocyanate, sodium iodide, sodium perchlorate, or urea.

18. The saliva stabilizing formulation of claim 14, wherein the biocide is 5-chloro-2-methyl-4-isothiazolin-3-one, sodium azide, 2-methyl-4-isothiazolin-3-one, thimerosal, or hypochlorite.

19. The saliva stabilizing formulation of claim 14, wherein the saliva stabilizing formulation stabilizes the DNA in the saliva for at least 7 days at room temperature.
